SEQUENCE LISTING (2 sequences)

SEQ ID NO: 1 - RNA, 15 nt, Artificial Sequence
  Synthetic
  modified_base (15)..(15): Biotin
uagguaguaguuuua

SEQ ID NO: 2 - RNA, 15 nt, Artificial Sequence
  Synthetic
  modified_base (15)..(15): Biotin
auugcacuccuuuua

(19) United States
(12) Patent Application Publication
(10) Pub. No.: US 2022/0291209 A1
(43) Pub. Date: Sep. 15, 2022

(54) MOLECULAR TARGET IN CANCER

(71) Applicant: The Regents of the University of California, Oakland, CA (US)

(72) Inventor: Tannishtha Reya, La Jolla, CA (US)

(21) Appl. No.: 17/647,342

(22) Filed: Jan. 6, 2022

Related U.S. Application Data
(60) Provisional application No. 63/134,943, filed on Jan. 7, 2021.

(51) Int. Cl. G01N 33/542; G01N 33/58; G01N 21/64; C07K 14/36; A61P 35/02

(52) U.S. Cl. CPC G01N 33/542; G01N 33/582; G01N 21/6428; C07K 14/36; A61P 35/02; C07K 2319/21

(57) ABSTRACT

Disclosed are methods for identifying small molecule modulators of protein-RNA interactions that could potentially function as therapeutic agents in cancer treatment. Methods of identifying inhibitors of the RNA-binding functions of MSI2, as well as inhibitors identified thereof, are also disclosed.

### PRIORITY CLAIM

This application claims priority to U.S. Provisional Application No. 63/134,943, filed Jan. 7, 2021, the contents of which are hereby incorporated by reference in its entirety.

### STATEMENT REGARDING SPONSORED RESEARCH

This invention was made with government support under CA197699 awarded by the National Institutes of Health. The government has certain rights in the invention.

### REFERENCE TO SEQUENCE LISTING SUBMITTED VIA EFS-WEB

This application includes an electronically submitted sequence listing in .txt format. The .txt file contains a sequence listing entitled “009062-8421.US02_ST25.txt” created on Apr. 25, 2022 and is 817 bytes in size. The sequence listing contained in this .txt file is part of the specification and is hereby incorporated by reference herein in its entirety.

### TECHNICAL FIELD

This patent document relates to the identification of compounds targeting protein-RNA interactions and the development of novel anti-cancer therapeutic agents and regenerative medicine drugs.

### BACKGROUND

Protein-RNA interactions are vital to many biological and pathological processes and functions. Understanding protein-RNA interactions and identifying modulators of such interactions are thus important to the identification and development of novel, effective therapeutic strategies for a number of human diseases.

In particular, Musashi RNA Binding Protein 2 (MSI2) is an RNA-binding protein (RBP) involved in stemness and is emerging as an important regulator of multiple critical biological processes relevant to cancer initiation, progression, and drug resistance. MSI2 binds and regulates the mRNA stability and translation of proteins operating in essential oncogenic signaling pathways, including NUMB/Notch. Many types of cancer involving dysfunction of the MSI2 pathway, including pancreatic cancer, lung cancer, leukemia, and colon cancer, are highly resistant to current treatments and thus require identification and development of novel, more effective therapeutic strategies, including those that target the MSI2 pathway.

### SUMMARY

This patent document discloses methods, compositions, and devices for the identification of small molecule modulators of protein-RNA interactions that could potentially function as therapeutic agents in cancer treatment. In particular, methods of identifying inhibitors of the RNA-binding functions of MSI2, as well as inhibitors identified thereof, are disclosed.

In one aspect, the present patent document provides a method of identifying a modulator such as an inhibitor or an enhancer of protein-RNA interactions, for example, MSI2-RNA interactions. In some embodiments, the identification is carried out by a Fluorescence Resonance Energy Transfer (FRET)-based assay, wherein the protein is coupled to a fluorophore donor and the RNA is coupled to a fluorophore acceptor. In some embodiments, the identification is carried out by a Fluorescence Resonance Energy Transfer (FRET)-based assay, wherein the RNA is coupled to a fluorophore donor and the protein is coupled to a fluorophore acceptor. In some embodiments, the method comprises: (a) detecting a first FRET signal generated by the protein-RNA interaction; (b) introducing an agent; and (c) detecting a second FRET signal. If the FRET signal decreases in the presence of the agent, the agent is an inhibitor of the protein-RNA interaction. If the FRET signal increases in the presence of the agent, the agent is an enhancer of the protein-RNA interaction. In some embodiments, the method is carried out in a high-throughput screening where multiple agents can be tested.

In some embodiments, disclosed herein is a method of identifying a modulator of protein-RNA interaction. The method entails (a) contacting a fluorophore-labeled RNA-binding protein (RBP) with a fluorophore-labeled target RNA in a reaction mix, (b) detecting a first fluorescence signal generated by the interaction of the RBP and the target RNA, (c) introducing an agent into the reaction mix, (d) detecting a second fluorescence signal in the presence of the agent, wherein the agent is an inhibitor of the RBP-RNA interaction if the second fluorescence signal is decreased relative to the first fluorescence signal, and wherein the agent is an enhancer of the RBP-RNA interaction if the second fluorescence signal is increased relative to the first fluorescence signal. In some embodiments, the RBP is MSI2, or U1A. In some embodiments, the target RNA is an MSI2-binding RNA such as NUMB mRNA. In some embodiments, the RBP is labeled by coupling to a fluorophore donor and the target RNA is labeled by coupling to a fluorophore acceptor. In some embodiments, the RBP is labeled by coupling to a fluorophore acceptor and the target RNA is labeled by coupling to a fluorophore donor. In some embodiment, detecting the first fluorescence signal or detecting the second fluorescence signal is carried out by a FRET-based assay. In some embodiments, the FRET-based assay is a Homogeneous Time Resolved Fluorescence (HTRF) assay. In some embodiments, the target RNA is biotinylated at the 5′ end. In some embodiments, the target RNA is biotinylated at the 3′ end. In some embodiments, the 5′ biotinylated target RNA is coupled to streptavidin. In some embodiments, the streptavidin is labeled with XL665 or d2. In some embodiments, the RBP comprises a His tag. In some embodiments, the His tagged RBP is coupled to Terbium (Tb) cryptate via an anti-His antibody attached to Terbium (Tb) cryptate.

In another aspect, this disclosure relates to inhibitors of MSI2-RNA interaction identified by the method disclosed herein. In some embodiments, the inhibitors of MSI2-RNA interaction include 2-{[4-(2,5-dimethyl-1H-pyrrol-1-yl)phenyl]sulfanyl}acetic acid, 4-[2-(2,5-dimethyl-1H-pyrrol-1-yl)thiophene-3-carbonyl]-3,4-dihydro-2H-1,4-benzoxazine-2-carboxamide, 5-({3-phenyl-5H,6H,7H,8H-[1,2,4]triazolo[4,3-a]pyrazin-7-yl}methyl)-4H,7H-pyrazolo[1,5-a]pyrimidin-7-one, 1-{3-[2-({5,7-dimethyl-[1,2,4]triazolo[4,3-a]pyrimidin-3-yl}sulfanyl)acetyl]phenyl}pyrrolidin-2-one, N-(1-acetylpiperidin-4-yl)-4,5-dimethylthiophene-2-carboxamide, or a derivative thereof.

In another aspect, this disclosure relates to a method of treating cancer in a subject by administering to the subject an effective amount of one or more of the inhibitors of RBP-RNA interactions such as the inhibitors of MSI2-RNA interactions identified by the method disclosed herein. In some embodiments, the cancer is an MSI2-expressing cancer such as pancreatic cancer, lung cancer, leukemia, or colon cancer. In some embodiments, the inhibitors of MSI2-RNA interaction include 2-{[4-(2,5-dimethyl-1H-pyrrol-1-yl)phenyl]sulfanyl}acetic acid, 4-[2-(2,5-dimethyl-1H-pyrrol-1-yl)thiophene-3-carbonyl]-3,4-dihydro-2H-1,4-benzoxazine-2-carboxamide, 5-({3-phenyl-5H,6H,7H,8H-[1,2,4]triazolo[4,3-a]pyrazin-7-yl}methyl)-4H,7H-pyrazolo[1,5-a]pyrimidin-7-one, 1-{3-[2-({5,7-dimethyl-[1,2,4]triazolo[4,3-a]pyrimidin-3-yl}sulfanyl)acetyl]phenyl}pyrrolidin-2-one, N-(1-acetylpiperidin-4-yl)-4,5-dimethylthiophene-2-carboxamide, or a derivative thereof.

### BRIEF DESCRIPTION OF THE DRAWINGS

This application contains at least one drawing executed in color. Copies of this application with color drawing(s) will be provided by the Office upon request and payment of the necessary fees.

FIGS. 1A and 1B show the results of MSI2:NUMB-CTRL competition assay and U1A:Target-CTRL competition assay, respectively. The data is shown on an absolute scale for the FRET ratio as calculated by the acceptor signal (=XL665) divided by the donor signal (=Tb cryptate) and multiplied by 10,000.

FIGS. 2A and 2B show the normalized results of MSI2:NUMB competition assay and U1A:Target competition assay, respectively. The data is shown on a relative scale for the FRET ratio, i.e., a normalized signal where 100% corresponds to full FRET signal, i.e., no competition and 0% corresponds to no FRET signal, i.e., full competition.

FIG. 3 shows the plate format of a 1536-well plate in a pilot screen.

FIGS. 4A and 4B show the preliminary results of MSI2:NUMB-Biotin5′ RNA binding assays HTRF. FIG. 4A: MSI2:NUMB-Biotin5′ RNA binding assay HTRF-Pilot Screen (n=1) BCN00000163 (1536 well plate). FIG. 4B: MSI2:NUMB-Biotin5′ RNA binding assay HTRF-Pilot Screen (n=2) BCN00000163 (1536 well plate).

FIGS. 5A and 5B show dose response (DR) studies of low and high activity compounds from BCN00000163 against U1A:Target RNA binding assay—AlphaScreen assay (1536 well plate). CPd #1 (original well: AA25), CPd #2 (original Well: H12)—Low values from compound hits (BCN00000163 plate). CPd #3 (original well: D6), CPd #4 (original well: D10)—high values from compound hits (BCN00000163 plate).

FIG. 6 illustrates the FRET assay principle.

FIG. 7 illustrates high throughput screening workflow.

FIG. 8 shows that the pilot screening on two days was highly reproducible.

FIG. 9 illustrates the assay performance during high throughput screening. The screening demonstrated high quality evidenced by very good assay statistics: average (z′)=0.90.

FIG. 10 demonstrates stable pharmacology shown by IC50 of RNA displacement on every assay plate. Four examples are shown.

FIG. 11 shows that structure replicates confirmed reproducibility.

FIG. 12 illustrates the pre-selection of compounds for IC50. (1) represents MSI2 selective, (2) represents U1A selective, and (3) represents unselective.

FIG. 13 shows the preliminary results from dose response testing. The green bullet represents the median value of the triplicates from confirmation run at 15 μM.

FIG. 14 shows the 10-point duplicate curves for 7 compounds which exhibited an IC50 less than 1 μM.

Table 1 shows the confirmation test results of the 1,391 compounds tested at a final compound concentration of 15 μM, with columns showing compound ID, compound structure (expressed in the simplified molecular-input line-entry system (SMILES)), MSI2 testing results of the triplicates and their median value (expressed as % inhibition of protein-RNA interaction), U1A testing results of the triplicates and their median value (expressed as % inhibition of protein-RNA interaction), and results from the primary screen, respectively. As shown in Table 1, certain compounds exhibited inhibitory effects on RNA binding to both MSI2 and U1A. These compounds have therapeutic effects due to their ability to inhibit function of RBPs in general.

Table 2 shows the dose response test results of the 512 compounds, with columns showing compound ID and the fitting results for the dose response data which are IC50 value (μM), slope value, min and max of the fit curve, goodness of fit (Rsquare), highest valid tested compound concentration, % inhibition value at highest valid tested compound concentration, dose response curve with data (rectangles) and fit (line), and compound structure (expressed in the simplified molecular-input line-entry system (SMILES)), respectively.

Table 3 shows a summary of the 512 compounds based on the dose response test results, with columns showing the compound ID, SMILES, compound structure, compound name, and normalized FRET ratio IC50 (μM).

Table 4 shows MSI2 inhibitory compounds with IC50 under 1 μM.

### DETAILED DESCRIPTION

MSI2 is an RNA-binding protein and has two N-terminal RNA-binding domains (RBDs), which can recognize and bind target mRNAs. One of MSI2's targets is NUMB, a negative regulator of the Notch signaling pathway. MSI2 protein can bind to NUMB mRNA and inhibit its translation, resulting in elevated Notch signaling, increased proliferation and survival, and decreased apoptosis of cancer cells.

### Methods of Identifying Modulators of MSI2-RNA Interactions

In some embodiments, the binding properties of MSI2 and NUMB RNA can be used to detect small molecule inhibitors of MSI2's RNA-binding functions in a FRET-based assay, such as Homogeneous Time Resolved Fluorescence (HTRF). FRET is based on the transfer of energy between two fluorophores, a donor and an acceptor, when in close proximity. If the MSI2 protein and a MSI2-binding RNA (e.g., NUMB mRNA) are coupled to a fluorophore donor and a fluorophore acceptor, respectively, or vice versa, the interaction of MSI2 and the RNA can bring the donor and the acceptor into proximity with each other, thus generating a FRET signal.

In some embodiments, a MSI2 binding assay was developed where streptavidin labeled with XL665 or d2 was used as the acceptor, and Terbium (Tb) cryptate was used as the donor. For example, in the MSI2 binding assay, NUMB RNA was biotinylated at the 5′ end to create NUMB-Biotin 5′ RNA, which was coupled to the XL665 or d2-labeled streptavidin acceptor through streptavidin-biotin interaction. The MSI2 protein was engineered to contain a His tag, which was coupled to the Tb cryptate donor through an anti-His antibody attached to the Tb cryptate. Due to the MSI2-NUMB RNA interaction, the XL665 or d2 acceptor was brought into proximity of the Tb cryptate donor, thus generating a detectable FRET signal. Parameters of the binding assay, including the concentrations of MSI2 and NUMB-Biotin 5′ RNA, were optimized. Thus, the interaction of the MSI2 protein to NUMB-Biotin 5′ RNA could create a detectable FRET signal that can be used for subsequent binding assays and screening for modulators of MSI2-RNA interactions.

In some embodiments, a competition assay was developed based on the above disclosed MSI2-NUMB RNA interaction and another RNA-binding protein, U1A, that has different target RNAs from MSI2. In the competition assay, a non-biotinylated NUMB RNA (NUMB-CTRL RNA) could compete with NUMB-Biotin 5′ RNA to bind the MSI2 protein. But because NUMB-CTRL RNA was not biotinylated and thus not coupled to the acceptor, it could not generate a FRET signal even when bound to the Tb cryptate-coupled MSI2 protein. As a result, NUMB-CTRL RNA was used to compete with NUMB-Biotin 5′ RNA to displace the FRET signal generated by MSI2/NUMB-Biotin 5′ RNA interaction. Conversely, a U1A target RNA would not displace the FRET signal generated by MSI2/NUMB-Biotin 5′ RNA interaction, because the U1A target RNA had no binding affinities to MSI2.

Similarly, for the U1A-target RNA competition assay, a non-biotinylated version of the target RNA (Target-CTRL RNA) could compete with the biotinylated target RNA (Target-Biotin 5′ RNA), and thus displace the binding signal of U1A/Target-Biotin 5′ RNA interaction. On the other hand, NUMB-CTRL RNA—because it would not interact with the U1A protein-did not affect the binding signal between U1A and Target-Biotin 5′ RNA.

In some embodiments, the binding assay was developed to identify small molecules that modulate the interaction between MSI2 and NUMB RNA, which could represent novel modulators of MSI2's abilities to interact with RNAs. If the FRET signals decreased when a small molecule was introduced to the reaction mix, it suggests that the small molecule is an inhibitor of MSI2 and inhibits the ability of MSI2 to bind its target RNAs. Thus, the assay provides an efficient means to identify modulators of MSI2 function, including inhibitory compounds, in a screening assay. Similarly, the binding assay was used to identify inhibitors of U1A, reflected by a disruption of the U1A-Target RNA interaction and a decrease in the FRET signal. As a result, the screening can identify inhibitory compounds targeting the RNA-binding functions of U1A (a pro-oncogenic pathway) and serve as a platform technology to systematically target the functions of other RNA-binding proteins, which could potentially serve as anti-cancer therapeutic agents.

In some embodiments, the MSI2 and NUMB-Biotin 5′ RNA binding assay can be carried out in a large scale for robust, sensitive, cell-free, high-throughput screening (HTS) of small molecules that prevent MSI2-NUMB RNA interactions. For example, the binding assay can be performed in a 1536-well format, utilizing TR-FRET technology and miniaturized, full libraries of small molecules. Hits generated from the HTS assay (e.g., with an inhibition level of greater than 30%) can be further analyzed by cellular assays for MSI2 function.

In some embodiments, the binding assay disclosed herein can be used to identify enhancers of MSI2-RNA interactions, which is reflected by an increase in the FRET signal. Therefore, the binding assay can be used for the discovery of novel MSI2 function modulators, including both inhibitors and enhancers of MSI2's interaction with RNAs. Therefore, embodiments of the presently disclosed technology could lead to the development of effective therapies for cancer treatment involving the MSI2 pathway.

It should be understood that the binding assay and the competition assay disclosed herein, whether performed in a laboratory scale or by high-throughput screening, is applicable to the study of other MSI2-interacting RNAs as well. Furthermore, the binding assay and the competition assay can be used to study other protein-RNA interactions and identify modulators of such interactions, which is not limited to the MSI2 protein.

### Compounds Identified as MSI2 Inhibitors

In some embodiments, inhibitory compounds of MSI2-RNA interactions were identified using the methods disclosed herein. In a primary screening of small molecule compound libraries using the MSI2 binding TR-FRET assay, 194,914 compounds were screened at 15 μM final concentration for induced inhibition of MSI2-RNA interaction. Hit compounds were selected with a cut-off of at least 30% inhibition. Subsequently, after analysis of donor- and acceptor-channels, and removal of potential detection artefacts, 1,391 compounds were selected and confirmed by MSI2 and U1A TR-FRET assays in triplicates and at 15 μM concentration (Table 1).

In some embodiments, 512 compounds were selected from the 1,391 compounds for further dose response testing. The 512 compounds were selected based on their drug-likeness, including structural characteristics, molecular properties, and polar surface areas. The dose response testing was performed for each of the 512 compounds in the MSI/NUMB RNA binding assay (Tables 2 and 3).

In particular, of the 512 compounds tested, 5 compounds demonstrated an IC50 value of less than 1 μM (Table 4).

The technology disclosed herein is an effective high-throughput identification of small molecule inhibitors of RNA-binding proteins, including MSI2. Considering the important roles MSI2 plays in human health and disease, including pancreatic cancer, lung cancer, leukemia, and colon cancer, the inhibitory compounds of MSI2 identified herein have great potential to serve as anti-cancer drugs and can lead to the development of novel cancer therapies.

While this patent document contains many specifics, these should not be construed as limitations on the scope of any invention or of what may be claimed, but rather as descriptions of features that may be specific to particular embodiments of particular inventions. Certain features that are described in this patent document in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. Moreover, the separation of various system components in the embodiments described in this patent document should not be understood as requiring such separation in all embodiments.

Only a few implementations and examples are described, and other implementations, enhancements and variations can be made based on what is described and illustrated in this patent document

The following examples are intended to illustrate various embodiments of the invention. As such, the specific embodiments discussed are not to be constructed as limitations on the scope of the invention. It will be apparent to one skilled in the art that various equivalents, changes, and modifications may be made without departing from the scope of invention, and it is understood that such equivalent embodiments are to be included herein. Further, all references cited in the disclosure are hereby incorporated by reference in their entirety, as if fully set forth herein.

### Example 1 Development of MSI2-NUMB 5′Biotin HTRF Binding Assay

A binding assay for MSI2 to NUMB-Biotin5′ RNA was developed and the following conditions were optimized: concentrations for MSI2, NUMB-Biotin5′ RNA binding, competing with NUMB-CTRL RNA for identifying the optimal concentration to displace binding signal between MSI2+NUMB-Biotin5′ RNA, optimizing binding assay parameters for U1A binding to target RNA-Biotin5′ for counter screen, and miniaturizing the binding assay to 1536 well format for HTS screening.

Reagents and protein/peptide: MSI2—LS Bio G2396 (provided in 20 mM Tris-HCL pH 8.0, 150 mM NaCl, 10% glycerol, 1 mM DTT); U1A—LS Bio G19194-10 (provided in 16 mM HEPES, pH 7.6, 400 mM NaCl, 20% glycerol); NUMB 5′Biotin—(IDT); NUMB 5′—(IDT); U1A RNA 5′ Biotin—(IDT); U1A RNA 5′—(IDT); HEPES pH 7.3 (RNase/DNase free)—(Fisher-BP299); Molecular grade water (RNase/DNase free)—(Invitrogen—10977-95); DTT (molecular grade—RNase/DNase free)—(Promega—V3151); Pluronic F-127—(Sigma Aldrich SKU #P2443); NaCl (RNase/DNase free)—(Sigma—S3014); 6His Terbium (TB) Cryptate—(CisBio—61HI2TLA); Streptavidin XL665 (CisBio—610SAXLA); AlphaPlate-1536 well flat bottom plate, Light gray (Perkin Elmer #6004350); and AlphaScreen Histidine Detection Kit (Perkin Elmer, Cat. No. 6760619C) containing AlphaScreen Nickel Chelate Acceptor Beads (5 mg/ml), and AlphaScreen Streptavidin Donor Beads (5 mg/ml).

MSI2:Numb/U1A:Target RNA Competition assay—HTRF (384 well plate): CisBio HTRF protocol was followed in a total assay volume of 20 μl. 5 uL of NUMB-CTRL RNA/Target-CTRL (4× Conc.) titrated in 10 point in 2× dilution, 5 uL of MSI2 protein/U1A protein (4× Conc.) final concentration of 5 nM was added to each well, 5 uL of NUMB-Biotin5′/Target-Biotin5′ RNA (4× Conc.) final concentration of 1.25 nM and Streptavidin XL665 acceptor (4×) final concentration of 5 nM were added, and incubated for 60 minutes at room temperature. 5 uL of 4× His TB cryptate, final concentration of 0.6 nM was added and incubated at room temperature for 60 minutes and read on envision. The assay buffer contained 150 mM NaCl, 25 mM HEPES pH 7.3, 1 mM DTT, and 0.05% pluronic F-127.

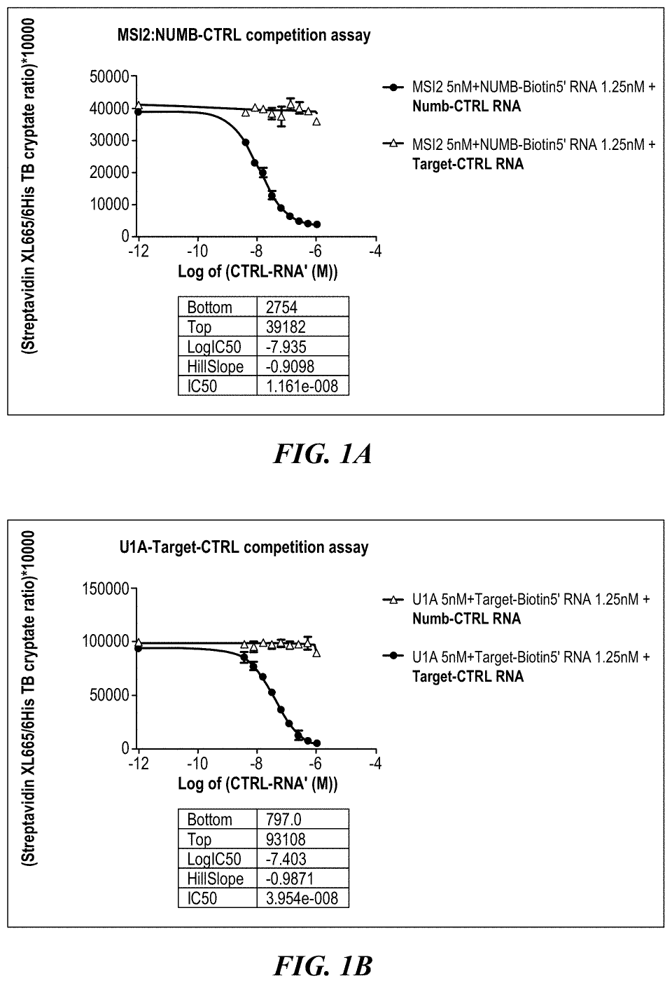

NUMB RNA or U1A target RNA was titrated starting from 1000 nM in 2× dilution over 9 points against MSI2 5 nM+NUMB-Biotin5′ 1.25 nM. FIG. 1A shows that the competition assay using NUMB RNA displaced the binding signal between MSI2 protein and NUMB-Biotin5′ RNA. In contrast, U1A target RNA did not displace the binding signal between MSI2 protein and NUMB-Biotin5′ RNA. NUMB RNA or U1A target RNA was titrated starting from 1000 nM in 2× dilution over 9 points against U1A 5 nM+U1A Target-Biotin5′ 1.25 nM. FIG. 1B shows that the competition assay using U1A target RNA displaced the binding signal between U1A protein and U1A target-Biotin5′ RNA. In contrast, NUMB RNA did not displace the binding signal between U1A protein and U1A target-Biotin5′ RNA. X axis: Log of CTRL RNA (M); Y axis: (Streptavidin XL665/6His TB cryptate ratio)*1000.

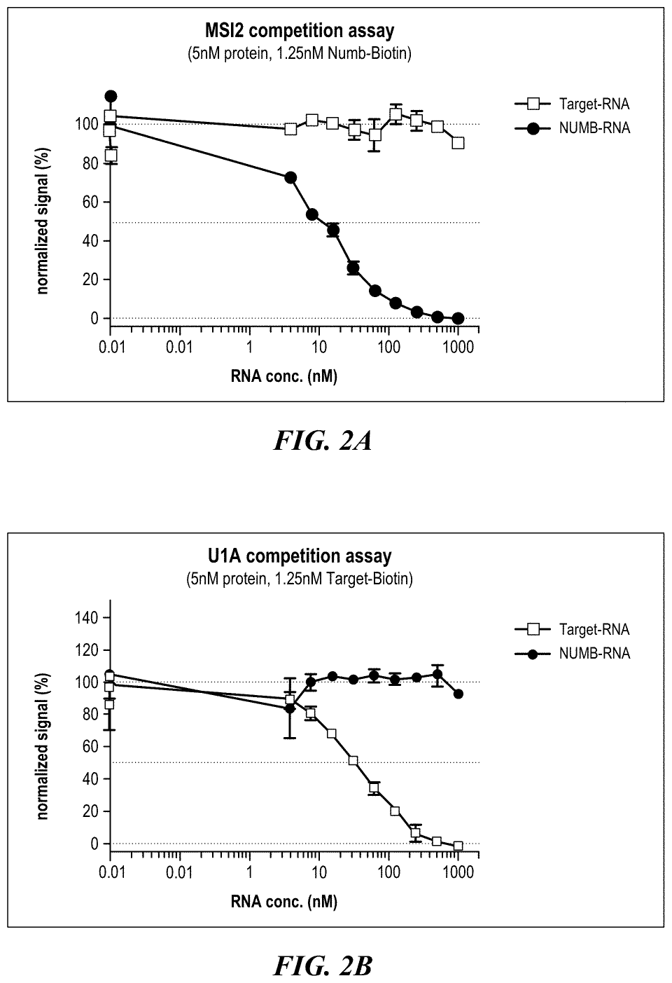

NUMB RNA or U1A target RNA was titrated starting from 1000 nM in 2× dilution over 9 points against MSI2 5 nM+NUMB-Biotin5′ 1.25 nM. FIG. 2A shows that the competition assay using NUMB RNA displaced the binding signal between MSI2 protein and NUMB-Biotin5′ RNA. In contrast, U1A target RNA did not displace the binding signal between MSI2 protein and NUMB-Biotin5′ RNA. NUMB RNA or U1A target RNA was titrated starting from 1000 nM in 2× dilution over 9 points against U1A 5 nM+U1A Target-Biotin5′ 1.25 nM. FIG. 2B shows that the competition assay using U1A target RNA displaced the binding signal between U1A protein and U1A target-Biotin5′ RNA. In contrast, NUMB RNA did not displace the binding signal between U1A protein and U1A target-Biotin5′ RNA. X axis: RNA concentration (nM); Y axis: % normalized signal.

MSI2:NUMB-Biotin5′ RNA assay—BCN00000163 pilot screen 1536 Well plate: CisBio HTRF protocol was followed in a total assay volume of 8 μl. The final compound concentration was 7.5 μM. All solutions were dispensed on Tempest. 2 uL assay buffer, 30 nL of DMSO or 2 mM compound (final concentration of 7.5 uM in assay 8 uL volume) in DMSO using BRAVO Pintool were added to each well, 2 uL of MSI2 protein (4× Conc.) final concentration of 5 nM was added to each well, 2 uL of NUMB-Biotin5′ RNA (4× Conc.) final concentration of 1.25 nM+Streptavidin XL665 acceptor (4×) final concentration of 5 nM were added, and incubated for 60 minutes at room temperature. For controls 2 uL of NUMB-CTRL RNA (4× Conc.) of 500 nM+NUMB-Biotin5′ RNA (4× Conc.) final concentration of 1.25 nM were added. 2 uL of 4× His TB cryptate, final concentration of 0.6 nM was added, and incubated at room temperature for 60 minutes and read on envision. The plates were centrifuged at 1000G for 1 minute after every dispense. The assay buffer contained 150 mM NaCl, 25 mM HEPES pH 7.3, 1 mM DTT, and 0.05% pluronic F-127.

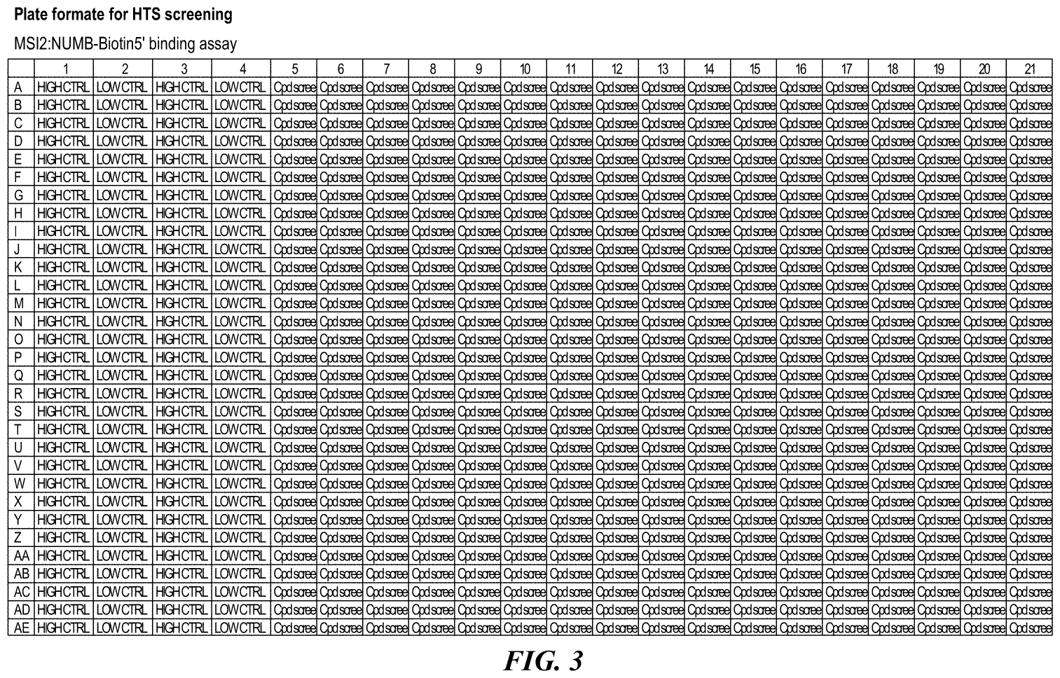

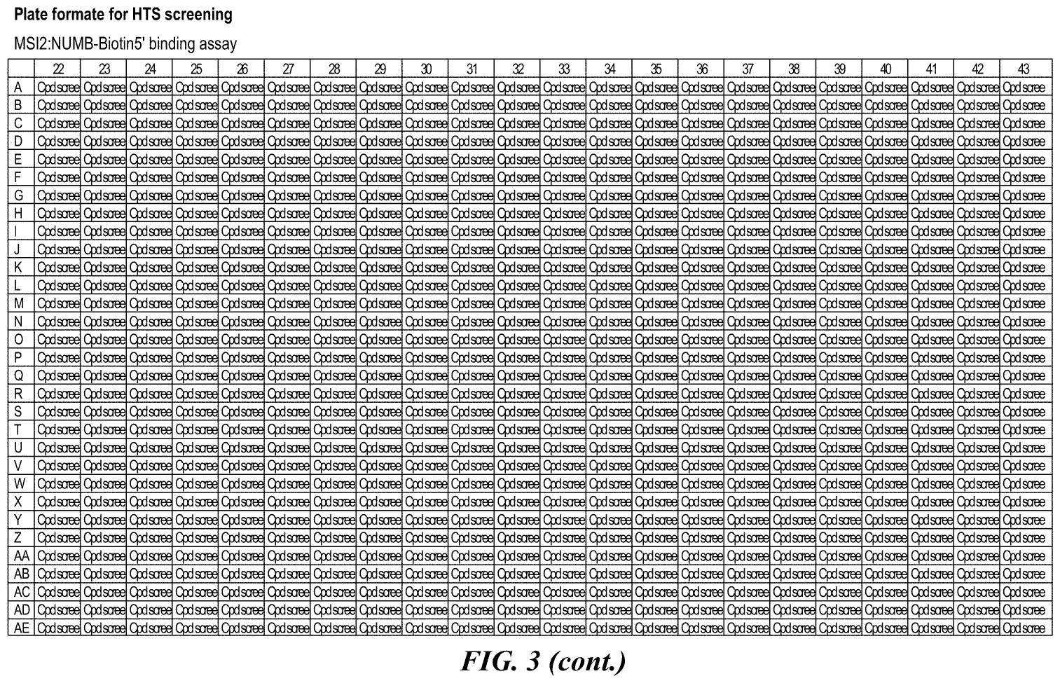

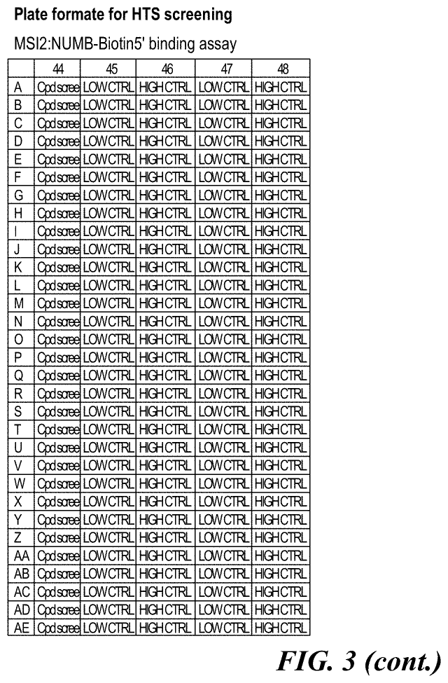

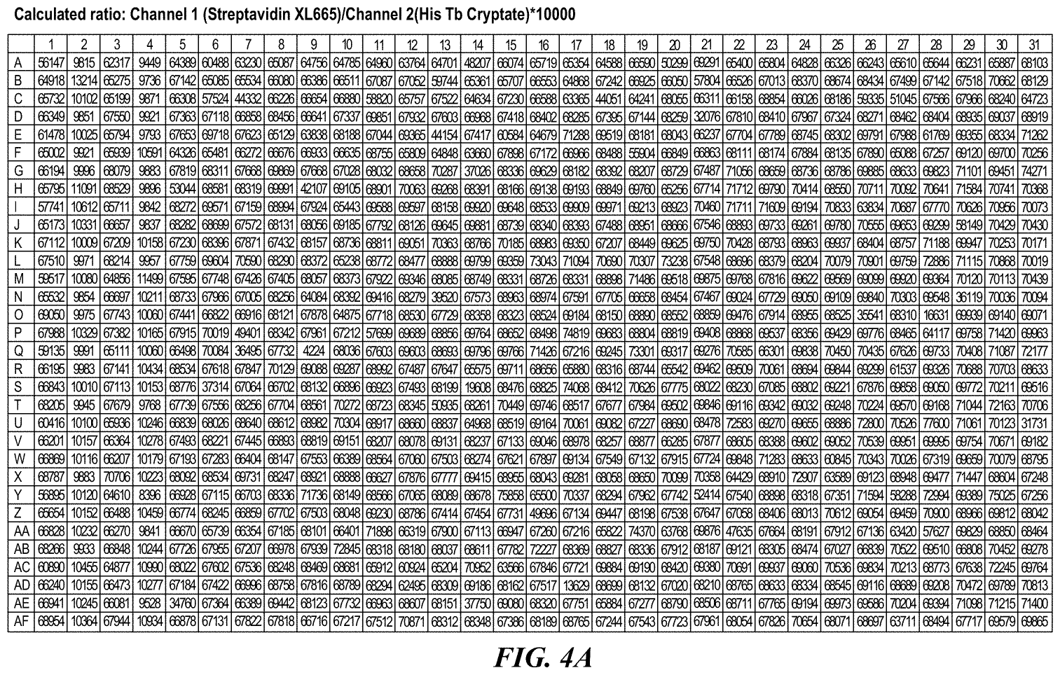

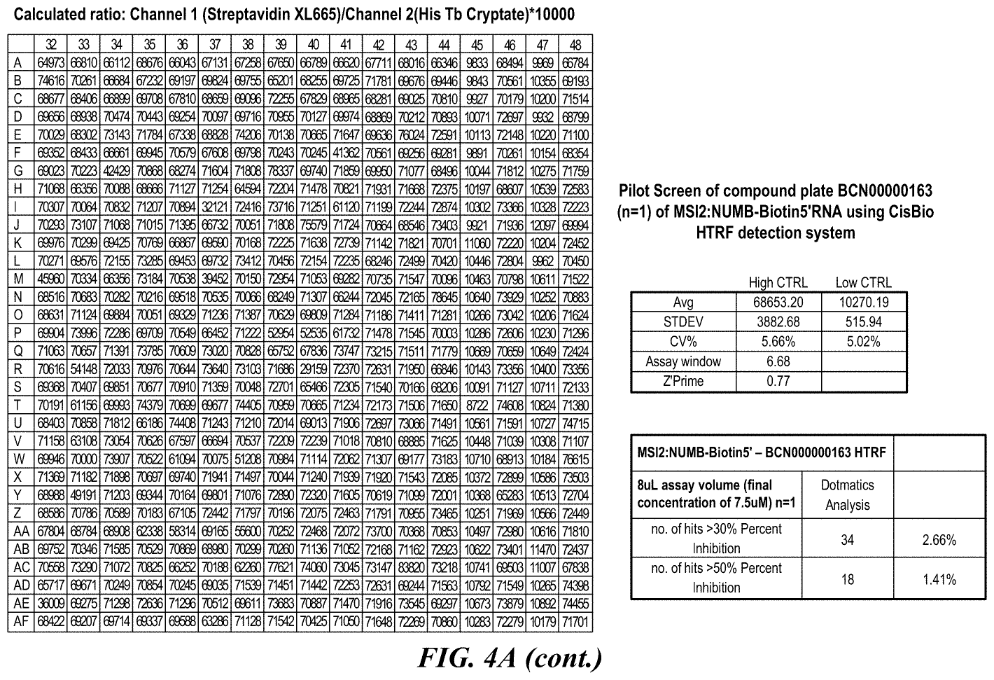

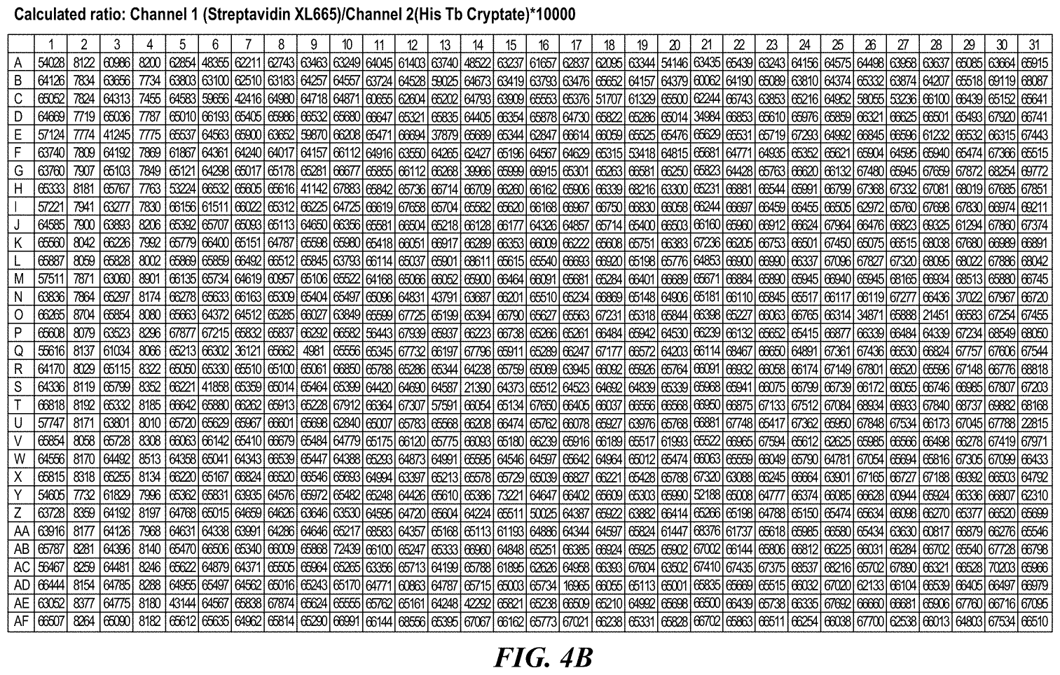

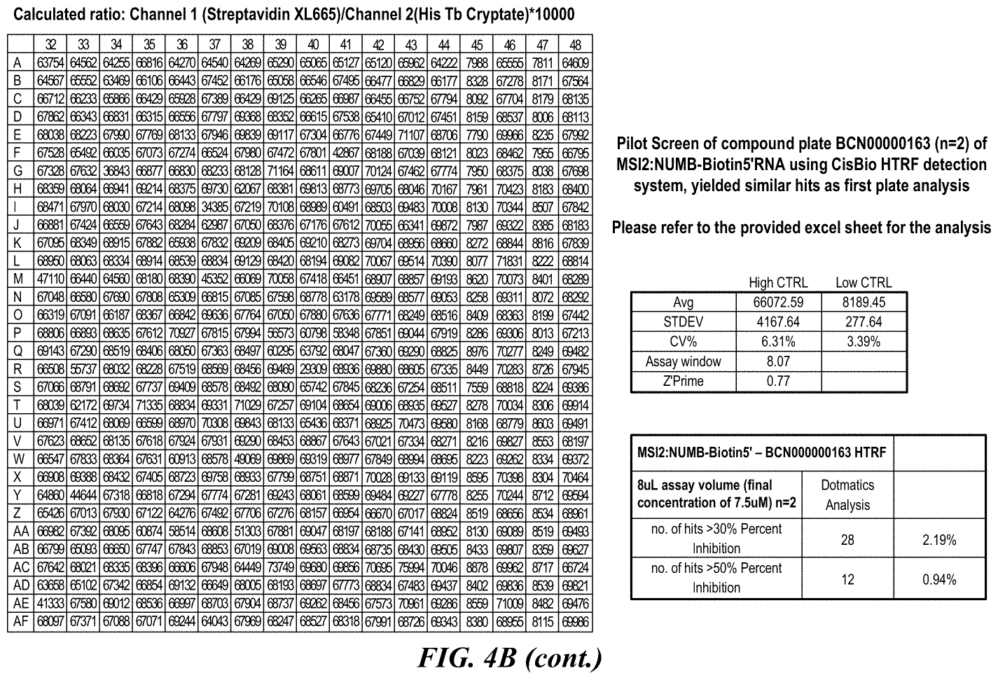

FIG. 3 illustrates an example of the plate format and FIG. 4 shows the preliminary results.

As demonstrated above, the competition assays of MSI2:NUMB-CTRL and U1A:Target-CTRL RNA displaced the FRET signal between the binding of the RBP and its target biotinylated RNA. However, MSI2:Target-CTRL or U1A:NUMB-CTRL did not displace the FRET signal between the binding of the RBP and its target biotinylated RNA. The MSI2 pilot screen of BCN00000163 twice demonstrated reasonable hits and compound hits match from both the runs of the same compound plate with assay window of 7 and Z'Prime of 0.77. The assay was miniaturized and optimized using HTRF (XL665 acceptor) and tested against pilot screen.

U1A:Target-Biotin5′ RNA assay dose response of compounds from BCN00000163 compound plate—AlphaScreen assay (1536 Well plate): The total assay volume was 10 μl and the final compound concentration was 7.5 μM. The compounds were dispensed on the ECHO acoustic dispenser and protein+RNA+beads dispensed on Tempest. Specifically, the compounds were dispensed using ECHO, titrated in 10 points of 3× dilution from 10 uM starting concentration, 3 uL assay buffer was added, 3 uL of U1A protein (3.33× Conc.) final concentration of 5 nM was added to each well, and incubated for 30 minutes at room temperature. 3 uL of Target-Biotin5′ RNA (4× Conc.) final concentration of 1.25 nM, +/−NUMB-CTRL RNA (3.33×) final concentration of 500 nM were added, 1 uL of 10× of streptavidin donor beads (final concentration 5 ug/ml)+Nickel chelator acceptor beads (final concentration 5 ug/ml) were added, and incubated at room temperature for 60 minutes and read on envision. The plates were centrifuged at 1000G for 1 minute after every dispense. The assay buffer contained 150 mM NaCl, 25 mM HEPES pH 7.3, 1 mM DTT, and 0.05% pluronic F-127.

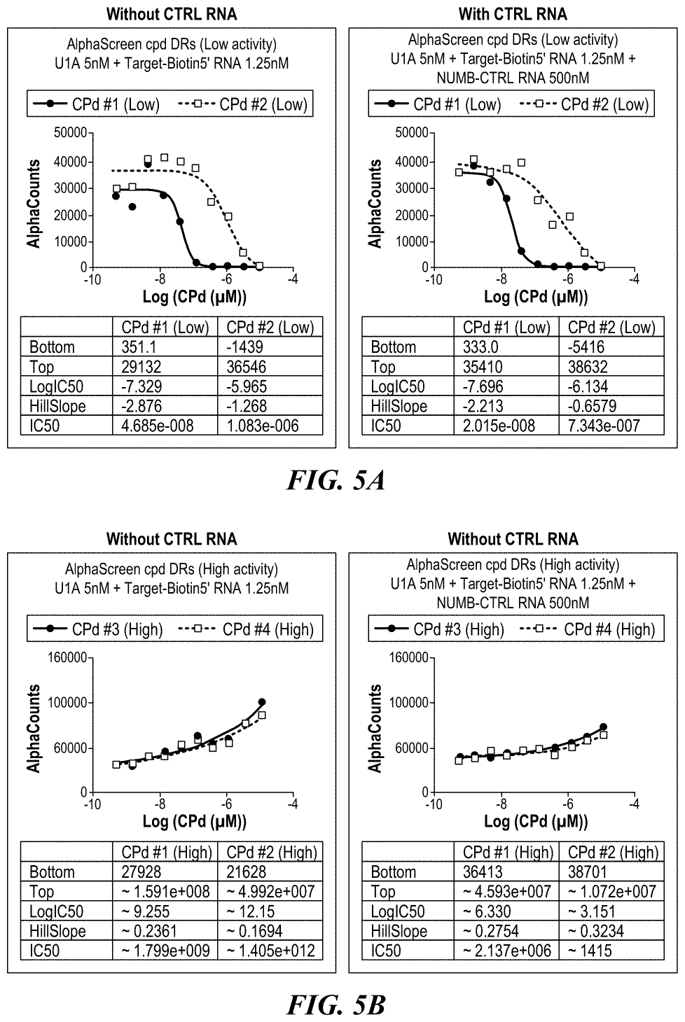

The compound dose response study was done with U1A:Target binding assay or without the NUMB-CTRL RNA. FIG. 5 shows that dose response of compounds which demonstrated hits or lowered activity (see CPd #1 and CPd #2, FIG. 5A) and which exhibited increased activity (see CPd #3 and CPd #4, FIG. 5B).

### Example 2 Development of High Throughput Screening (HTS) of MSI2 Assay

Robust and sensitive, cell-free protein-RNA interaction assays with TR-FRET readout for MSI2 and another RNA binding protein (U1A) were designed and implemented. Miniaturized, full library high throughput screening was performed to identify small molecules that prevent protein-RNA interaction.

The screening cascade included HTS assays and secondary assays. The HTS assays included primary assays probing MSI2-RNA inhibition and profiling assays probing U1A-RNA inhibition. The secondary assays included cellular assays for MSI2 function and biophysical methods to validate FRET, showing target engagement i.e. direct interaction of small molecule with protein.

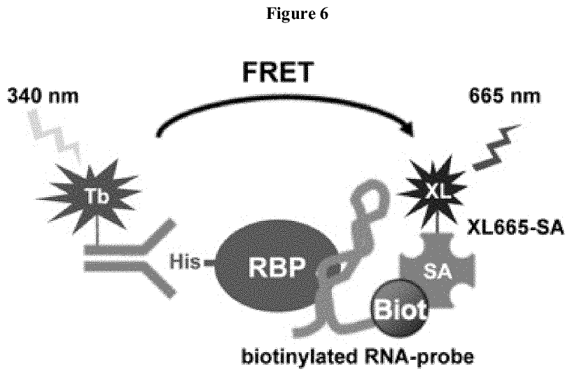

FIG. 6 illustrates the FRET assay principle for MSI2 and U1A. The NUMB RNA probe for MSI2 was selected based on prior scientific publications and has the following sequence: 5′UAGGUAGUAGUUUUA-Biotin (SEQ ID NO: 1)(Oncotarget, 8(63): 106587-106597 (2017)). The RNA probe for U1A was designed based on an interaction study between U1A and RNA done by NMR (Structure 4(5): 621-631 (1996)) and has the following sequence: 5′AUUGCACUCCUUUUA-Biotin (SEQ ID NO: 2). The underlined sequence represents the part which interacts with the protein, the sequence of AYYGCAC should fit into the groove of U1A according to the NMR studies. The remaining sequence without underline is a spacer. The assay developed provides robust TR-FRET readout.

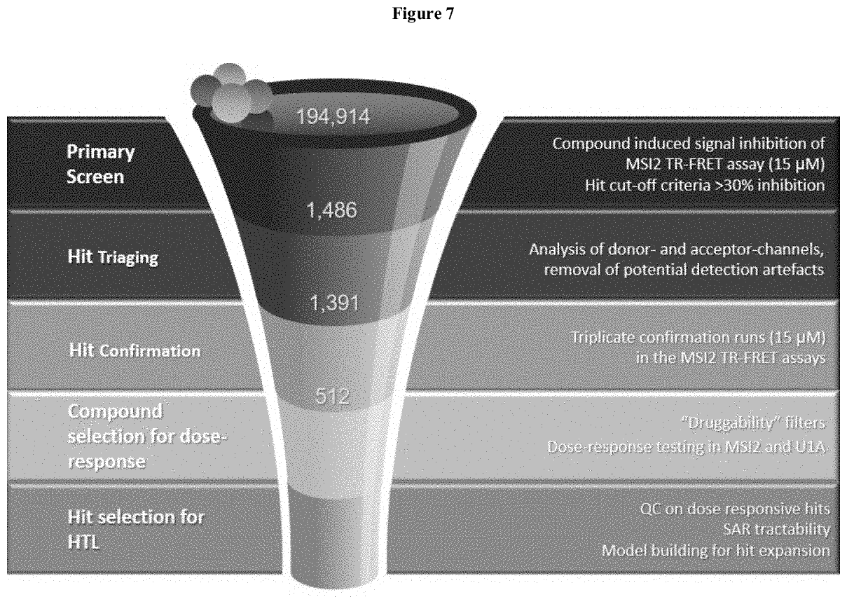

FIG. 7 illustrates the high throughput screening workflow. The primary screen is based on compound induced signal inhibition of MSI2 TR-FRET assay (15 μM). The hit cut-off criteria are >30% inhibition. The hit triaging is based on analysis of donor- and acceptor-channels, removal of potential detection artefacts. The hit confirmation is based on triplicate confirmation runs (15 μM) in MSI2 and U1A TR-FRET assays. The compound selection for dose-response is a dose-response testing in MSI2 and U1A to filter for compounds that have the drug potential. The hit selection for hit to lead (HTL) is based on QC on dose responsive hits SAR tractability, and model building for hit expansion.

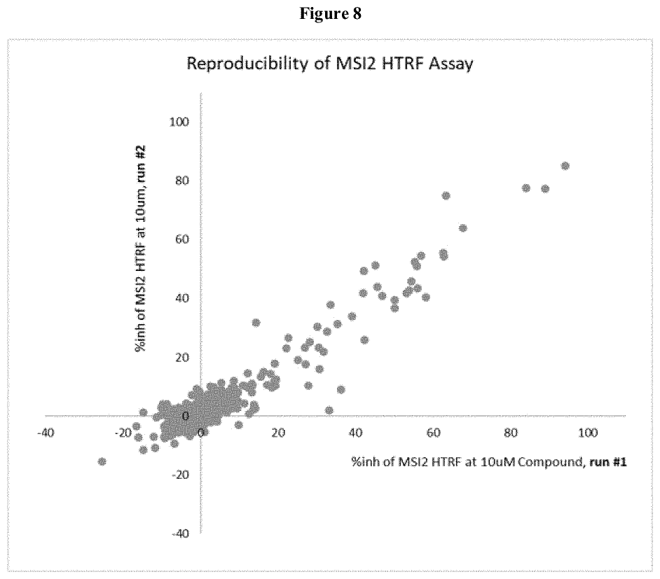

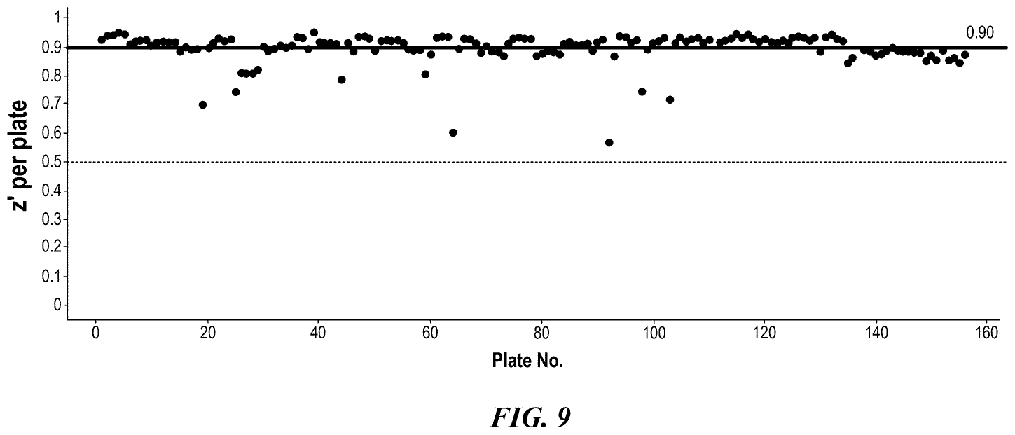

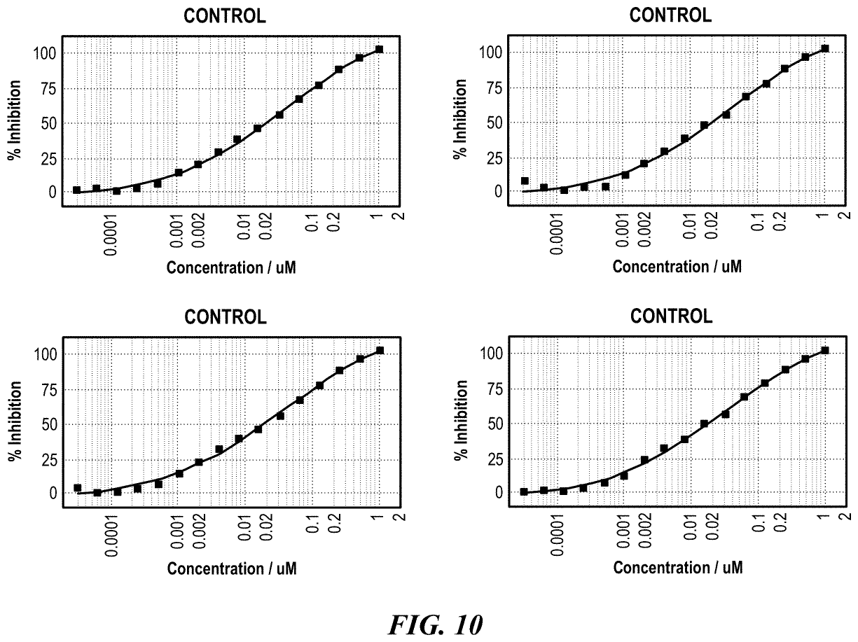

FIG. 8 shows that the pilot screening on two days was highly reproducible. FIG. 9 illustrates the assay performance during high throughput screening. FIG. 10 demonstrates stable pharmacology.

Table 1 shows the confirmation test results of the 1,391 compounds, with columns showing compound ID, compound structure (expressed in the simplified molecular-input line-entry system (SMILES)), MSI2 testing results of the triplicates and their median value (expressed as % inhibition of protein-RNA interaction), U1A testing results of the triplicates and their median value (expressed as % inhibition of protein-RNA interaction), and results from the primary screen, respectively.

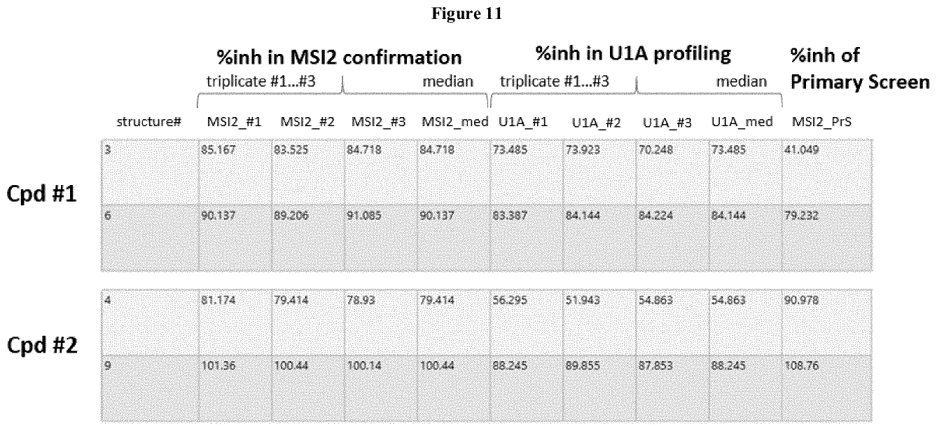

In the set of 1391 hit compounds, structure replicates different compound ID were identified as shown in FIG. 11. Findings for structure replicates further increased confidence in the data. The % inhibition of structure replicates in primary screen was very similar, given single point data. Triplicates of confirmation and profiling ran tight and confirmed the primary data.

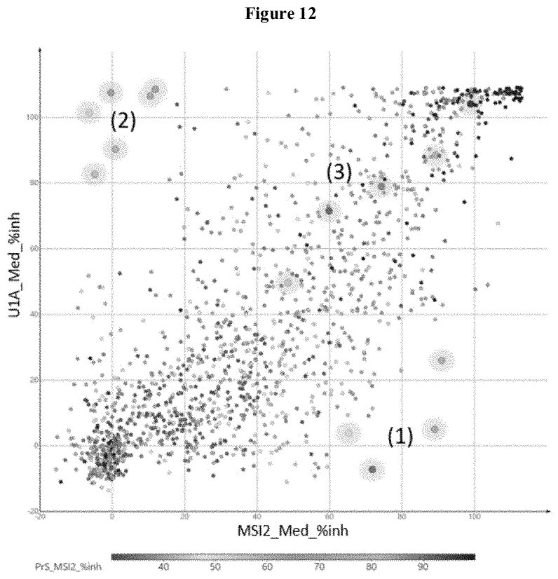

FIG. 12 illustrates pre-selection of compounds for IC50. The confirmation assay was performed as single concentration assays at 15 μM compound. The dose response experiments for these compounds were performed in MSI2 assay and in U1A assay.

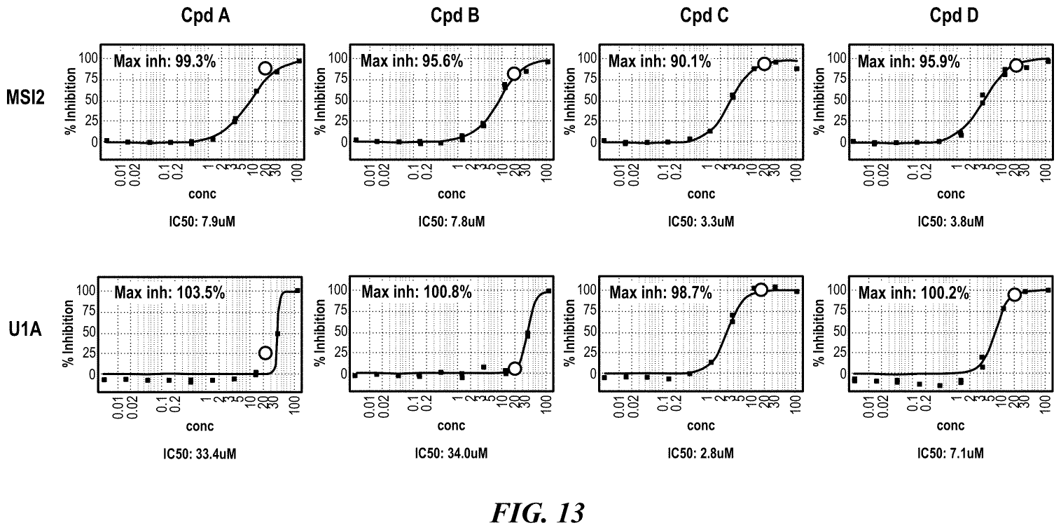

FIG. 13 shows the preliminary results from dose response testing. The dose response experiments reproduced confirmation assays. Cpd A and Cpd B are MSI2 selective compounds with low micromolar potency, e.g., 4 to 5-fold. Cpd C and Cpd D are unselective or slightly selective compounds with low micromolar potency.

This example demonstrates that sensitive and robust RNA-protein binding assays for MSI2 and U1A were established and validated. The full library small molecule screening yielded about 1400 hits. MSI2 selective compounds were identified based on preliminary dose response results. 256 compounds will be selected for dose response test for MSI2 and U1A. Structure activity relationship (SAR) analysis of the results and hit expansion will be performed. The subset of hits will be submitted to cell-based assay. Target engagement will be tested by orthogonal assays.

### Example 3 IC50 Analysis of 512 Compounds Selected from Screening

IC50 analysis of 512 compounds selected from the confirmation screen (MSI2:NUMB-Biotin5′) screen was performed. The 512 compounds were run in duplicate 10-point dose response curve with a high concentration of 100 uM and a low concentration of 5.6 nM. The total volume of the assay was 8 μl and all steps except for ECHO dispense were performed on Tempest disperser. 80 nL of compounds in DMSO was dispensed in 10 point titrations of 3× dilutions with the t concentration of 100 μM and the bottom concentration of 5.6 nM from 10 mM stock using ECHO acoustic dispenser—duplicate curves on the same assay plate. 2 μL of assay buffer was added, 2 μL of MSI2 (4× Conc.) final concentration of 5 nM was added, and incubated for 30 minutes. Low CTRLS: 2 μL of NUMB-CTRL (4× Conc.) final concentration of 1000 nM+NUMB-Biotin5′ (4× Conc.) final concentration of 1.25 nM+4× concentration of Streptavidin XL665 were added for a final concentration of 5 nM. QC Low CTRL wells: 80 nL of NUMB-CTRL (Titrated in 16 points at 2× dilution, starting from 1000 nM concentration—final concentration 0.03 nM) using ECHO 555 Acoustic dispenser. The RNA was titrated into 2 fold dilutions in water in ECHO LDV plates and used to dispense each assay plate for IC50 reference. Subsequently, 2 μL of NUMB-Biotin5′ (4× Conc.) final concentration of 1.25 nM+4× conc. of Streptavidin XL665 were added for a final concentration of 5 nM and incubated at room temperature for 15 minutes. 2 μL of 4× His TB-Cryptate was added to a final concentration of 0.6 nM and incubated for 1 hour at room temperature and read on envision. The assay buffer contained 150 mM NaCl, 25 mM HEPES pH 7.3, 1 mM DTT, and 0.05% pluronic F-127.

Table 2 shows the dose response test results of the 512 compounds, with columns showing compound ID, IC50 value (μM), slope value, dose response curve, and compound structure (expressed in the simplified molecular-input line-entry system (SMILES)), respectively.

Table 3 shows a summary of the 512 compounds based on the dose response test results, with columns showing the compound ID, SMILES, compound structure, compound name, and FRET ratio IC50 (μM). The FRET ratio was normalized on a % inhibition scale between 0% (that is, no effect of compound/no inhibition) and 100% (that is, maximum effect of a control/full inhibition). The lower the FRET ratio IC50, the more potent the compound is.

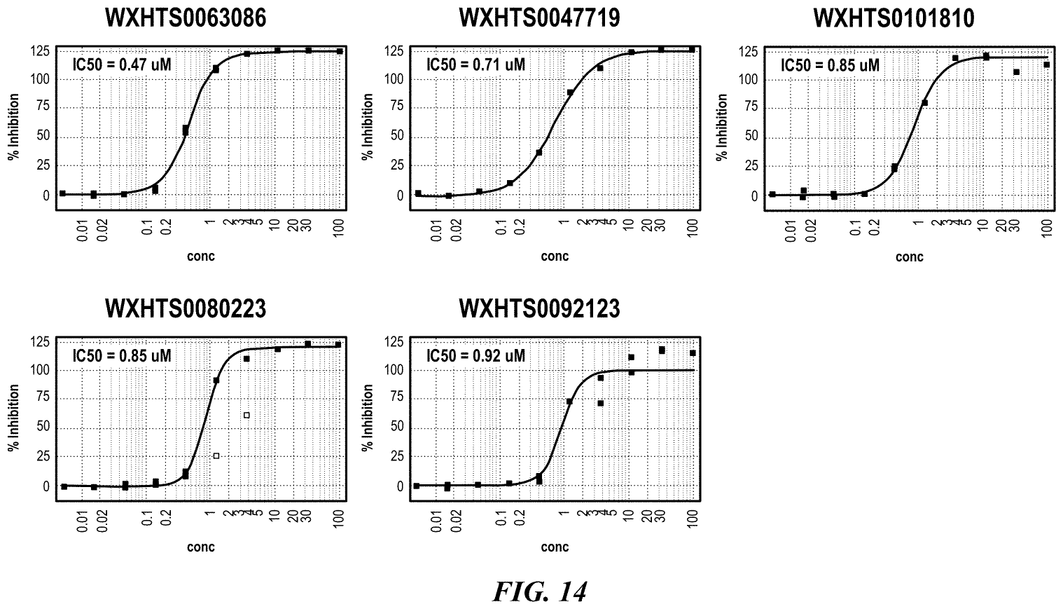

Among the 512 compounds, 490 compounds provided DRC curves within the concentration range specified, and 22 compounds were out of the specified DRC range. Among the 490 compounds within the specified range, 399 compounds had a slope less than 3.0, 89 compounds had a slope greater than 3.0, and 1 compound had a slope less than 0.5. Among the 490 compounds, 5 compounds demonstrated an IC50 of less than 1 μM (FIG. 14), and 14 compounds had an IC50 below or about 2 μM, shown in Table 4.

Lengthy table referenced here
US20220291209A1-20220915-T00001
Please refer to the end of the specification for access instructions.

Lengthy table referenced here
US20220291209A1-20220915-T00002
Please refer to the end of the specification for access instructions.

Lengthy table referenced here
US20220291209A1-20220915-T00003
Please refer to the end of the specification for access instructions.

Lengthy table referenced here
US20220291209A1-20220915-T00004
Please refer to the end of the specification for access instructions.

LENGTHY TABLES
The patent application contains a lengthy table section. A copy of the table is available in electronic form from the USPTO web site (https://seqdata.uspto.gov/?pageRequest=docDetail&DocID=US20220291209A1). An electronic copy of the table will also be available from the USPTO upon request and payment of the fee set forth in 37 CFR 1.19(b)(3).

1. A method of identifying a modulator of protein-RNA interaction comprising: (a) contacting a fluorophore-labeled RNA-binding protein (RBP) with a fluorophore-labeled target RNA in a reaction mix, (b) detecting a first fluorescence signal generated by the interaction of the RBP and the target RNA, (c) introducing an agent into the reaction mix, (d) detecting a second fluorescence signal in the presence of the agent, wherein the agent is an inhibitor of the RBP-RNA interaction if the second fluorescence signal is decreased relative to the first fluorescence signal, and wherein the agent is an enhancer of the RBP-RNA interaction if the second fluorescence signal is increased relative to the first fluorescence signal.

2. The method of claim 1, wherein the RBP is MSI2 or U1A.

3. The method of claim 1, wherein the target RNA is an MSI2-binding RNA.

4. The method of claim 1, wherein the target RNA is NUMB mRNA.

5. The method of claim 1, wherein the RBP is labeled by coupling to a fluorophore donor and the target RNA is labeled by coupling to a fluorophore acceptor.

6. The method of claim 1, wherein the RBP is labeled by coupling to a fluorophore acceptor and the target RNA is labeled by coupling to a fluorophore donor.

7. The method of claim 1, wherein detecting the first fluorescence signal or detecting the second fluorescence signal is carried out by a FRET-based assay.

8. The method of claim 7, wherein the FRET-based assay is a Homogeneous Time Resolved Fluorescence (HTRF) assay.

9. The method of claim 1, wherein the target RNA is biotinylated at the 5′ end or at the 3′ end.

10. The method of claim 9, wherein the biotinylated target RNA is coupled to streptavidin.

11. The method of claim 10, wherein the streptavidin is labeled with XL665 or d2.

12. The method of claim 1, wherein the RBP comprises a His tag.

13. The method of claim 12, wherein the His tagged RBP is coupled to Terbium (Tb) cryptate via an anti-His antibody attached to Terbium (Tb) cryptate.

14. An inhibitor of MSI2-RNA interaction identified by the method of claim 1, selected from the group consisting of 2-{[4-(2,5-dimethyl-1H-pyrrol-1-yl)phenyl]sulfanyl}acetic acid, 4-[2-(2,5-dimethyl-1H-pyrrol-1-yl)thiophene-3-carbonyl]-3,4-dihydro-2H-1,4-benzoxazine-2-carboxamide, 5-({3-phenyl-5H,6H,7H,8H-[1,2,4]triazolo[4,3-a]pyrazin-7-yl}methyl)-4H,7H-pyrazolo[1,5-a]pyrimidin-7-one, 1-{3-[2-({5,7-dimethyl-[1,2,4]triazolo[4,3-a]pyrimidin-3-yl}sulfanyl)acetyl]phenyl}pyrrolidin-2-one, N-(1-acetylpiperidin-4-yl)-4,5-dimethylthiophene-2-carboxamide, and a derivative thereof.

15. A method of treating cancer in a subject comprising administering to the subject an effective amount of one or more of the inhibitors of RBP-RNA interactions selected from the group consisting of 2-{[4-(2,5-dimethyl-1H-pyrrol-1-yl)phenyl]sulfanyl}acetic acid, 4-[2-(2,5-dimethyl-1H-pyrrol-1-yl)thiophene-3-carbonyl]-3,4-dihydro-2H-1,4-benzoxazine-2-carboxamide, 5-({3-phenyl-5H,6H,7H,8H-[1,2,4]triazolo[4,3-a]pyrazin-7-yl}methyl)-4H,7H-pyrazolo[1,5-a]pyrimidin-7-one, 1-{3-[2-({5,7-dimethyl-[1,2,4]triazolo[4,3-a]pyrimidin-3-yl}sulfanyl)acetyl]phenyl}pyrrolidin-2-one, N-(1-acetylpiperidin-4-yl)-4,5-dimethylthiophene-2-carboxamide, and a derivative thereof.

16. The method of claim 15, wherein the cancer is an MSI2-expressing cancer.

17. The method of claim 15, wherein the cancer is pancreatic cancer, lung cancer, leukemia, or colon cancer.
